(19) United States
(12) Patent Application Publication
(10) Pub. No.: US 2002/0028490 A1
(43) Pub. Date: Mar. 7, 2002

(54) Process for the production of L-amino acids by fermentation using coryneform bacteria

(76) Inventors: Douwe Molenaar, Baesweiler (DE); Michel Eduard Van Der Rest, Venlo (DE); Bettina Mockel, Bielefeld (DE)

Correspondence Address: PILLSBURY WINTHROP LLP, 1600 TYSONS BOULEVARD, MCLEAN, VA, 22102, US

(21) Appl. No.: 09/852,157

(22) Filed: May 10, 2001

Related U.S. Application Data
(63) Continuation-in-part of application No. 09/436,362, filed on Nov. 9, 1999, now abandoned.

(30) Foreign Application Priority Data
Mar. 19, 1999 (DE) 19912384.5

(51) Int. Cl. C12P 13/08; C12P 13/04; C12N 1/21

(52) U.S. Cl. 435/106; 435/252.3; 435/115

(57) ABSTRACT

The invention relates to a process for the production of L-amino acids by fermentation using coryneform bacteria in which the mqo gene is enhanced.

### FIELD OF THE INVENTION

The present invention provides an improved process for the production of L-amino acids, especially L-lysine, by fermentation using coryneform bacteria. The main characteristic of the process is the enhancement of bacterial malate:quinone oxidoreductase activity.

### BACKGROUND OF THE INVENTION

L-amino acids, especially L-lysine, are used in the feeding of animals, in human medicine and in the pharmaceutical industry. They are typically produced by fermenting strains of coryneform bacteria, especially Corynebacterium glutamicum. Because of the great importance of amino acids, work is continually being done to improve production processes. Improvements may concern measures relating to the fermentation process (e.g., relating to stirring and oxygen supply) or the composition of the nutrient medium, (e.g., relating to the sugar concentration during the fermentation). They may also concern the purification of product (e.g., by ion-exchange chromatography) or the intrinsic performance properties of the microorganism itself.

To improve the performance properties of amino acid-producing microorganisms, methods of mutagenesis, selection and mutant selection are often employed. These methods may be used to obtain strains that are resistant to antimetabolites, such as, for example, the lysine analogue S-(2-aminoethyl)-cysteine, or which are auxotrophic for amino acids which are important in terms of regulation, and produce L-amino acids. In addition, methods of recombinant DNA technology have been used to improve the L-amino-acid-producing strains of Corynebacterium glutamicum by amplifying individual genes of amino acid biosynthesis. General articles on this subject include Kinoshita (“Glutamic Acid Bacteria,” in: Biology of Industrial Microorganisms, Demain and Solomon (eds.), Benjamin Cummings, London, UK, 1985, 115-142; Hilliger, BioTec 2:40-44 (1991); Eggeling, Amino Acids 6:261-272 (1994); Jetten, et al., Crit. Rev. Biotech.15:73-103 (1995); and Sahm, et al., Ann. New York Acad. Sci. 782:25-39 (1996)).

### SUMMARY OF THE INVENTION

In its first aspect, the present invention is directed to an improvement in processes used to produce amino acids by fermenting bacteria of the coryneform genus. The improvement involves enhancing the activity of the malate:quinone oxidoreductase enzyme. Enhancement in this sense means increasing enzymatic activity above that seen in the unenhanced bacteria. Preferably, enhancement is accomplished by either amplifying the gene encoding the enzyme or by increasing the rate at which mRNA for the enzyme is transcribed. Techniques for amplification and for increasing transcription (e.g., by recombinantly transforming the bacteria with DNA encoding the enzyme and under the control of a strong promoter such as the CMV promoter) are well known in the art. One preferred vector for transforming bacteria is plasmid pRM17, which has been deposited in Corynebacterium glutamicum, under accession number DSM12711.

The bacteria that have undergone an enhancement of malate:quinone oxidoreductase activity may also undergo other alterations designed to increase amino acid production. For example, it is known that increased amino acid synthesis can be achieved by enhancing the activity of enzymes involved in synthetic pathways for amino acids, usually by over-expressing the gene encoding the enzyme. These approaches to increasing production may be combined with increasing malate:quinone oxidoreductase as discussed herein. Similarly the bacteria may be treated to eliminate one or more metabolic pathways that reduce the formation of the desired L-amino acid. Specific approaches that may be taken include over-expressing a gene coding for dihydrodipicolinate synthase or amplifying a DNA fragment mediating S-(2-aminoethyl)-cysteine resistance. The term “over-expressing” as used in this instance refers to treating bacteria so as to increase the amount of mRNA transcribed from a gene relative to the amount of transcription occurring in the untreated bacteria. Preferred amino acids for production by these methods are: L-aspartic acid, L-asparagine, L-homoserine, L-threonine, L-isoleucine and L-methionine, with the most preferred amino acid being L-lysine.

In a second aspect, the invention is directed to a process for producing an L-amino acid by the fermentation of bacteria of the coryneform genus, in which expression of the malate:quinone oxidoreductase gene is increased (e.g., by amplification of the gene). One way to enhance expression is by transforming the bacteria with the plasmid vector pRM17 as mentioned above. The bacteria are then fermented and amino acid is isolated using procedures known in the art. The bacteria may also be treated to enhance the activity of one or more additional enzymes of a synthetic pathway for an L-amino acid. For example, recombinant techniques may be used to enhance activity. Preferred amino acids for production using this procedure include L-aspartic acid, L-asparagine, L-homoserine, L-threonine, L-isoleucine and L-methionine. Of these, the most preferred is L-lysine.

### DETAILED DESCRIPTION OF THE INVENTION

The present invention is based upon the development of improved methods for the production of L-amino acids, especially L-lysine, by fermentation. Unless otherwise indicated, any mention of L-lysine or lysine herein is to be understood as meaning not only the base but also salt forms of the amino acid, such as, for example, lysine monohydrochloride or lysine sulfate. This also applies with respect to other amino acids.

The invention provides a process for the production of L-amino acids, especially L-lysine, by fermentation using coryneform bacteria which, especially, already produce the desired amino acid and in which the activity of the enzyme malate:quinone oxidoreductase (mqo) is enhanced, especially by over-expression of its gene. The term “enhanced” or “enhancement” in this connection describes a change which leads to an increase in the intracellular activity of the enzyme relative to the activity seen in the unaltered microorganism. For example, enhancement may be accomplished by increasing the copy number of the gene, using a strong promoter, or using a gene or allele that codes for a corresponding enzyme having a high degree of activity, and optionally combining those measures. “Amplification” refers to a specific procedure for achieving an enhancement whereby the number of DNA molecules carrying a gene or genes, an allele or alleles, a regulatory signal or signals or any other genetic feature is increased.

The microorganisms provided by the present invention can produce L-amino acids, especially L-lysine, from glucose, saccharose, lactose, fructose, maltose, molasses, starch, cellulose or from glycerol and ethanol. They are representatives of coryneform bacteria, especially of the genus Corynebacterium. In this genus, a preferred species is Corynebacterium glutamicum. Examples of suitable strains of bacteria are as follows:

| | | |
|---|---|---|
| Corynebacterium glutamicum | | ATCC13032 |
| Corynebacterium acetoglutamicum | | ATCC15806 |
| Corynebacterium acetoacidophilum | | ATCC13870 |
| Corynebacterium thermoaminogenes | FERM | BP-1539 |
| Brevibacterium flavum | | ATCC14067 |
| Brevibacterium lactofermentum | | ATCC13869 |
| Brevibacterium divaricatum | | ATCC14020. |

Examples of suitable L-amino-acid-producing, especially L-lysine-producing, mutants and strains produced therefrom, include:

| | | |
|---|---|---|
| Corynebacterium glutamicum | FERM-P | 1709 |
| Brevibacterium flavum | FERM-P | 1708 |
| Brevibacterium lactofermentum | FERM-P | 1712 |
| Brevibacterium flavum | FERM-P | 6463 |
| Brevibacterium flavum | FERM-P 6 | 464. |

The inventors have found that coryneform bacteria produce L-amino acids, especially L-lysine, in an improved manner after over-expression of malate:quinone oxidoreductase. The mqo gene codes for the enzyme malate:quinone oxidoreductase (EC 1.1.99.16), which catalyses the oxidation of malate to oxalacetate with transfer of the electrons to ubiquinone-1. The nucleotide sequence of the mqo gene of Corynebacterium glutamicum has been determined by Molenaar, et al. (Eur. J Biochem. 254:395-403 (1998)) and is available at the nucleotide sequence databank of the National Center for Biotechnology Information (NCBI, Bethesda, Md., U.S.A.) under accession number AJ 22 4946. In addition to the gene of C. glutamicum described by Molenaar et al., it is also possible to use alleles of the mqo gene which result from the degeneracy of the genetic code or by function-neutral sense mutations.

In order to achieve over-expression, the copy number of the mqo gene can be increased, or the promoter and regulation region, which is located in front of the structural gene, can be mutated. Expression cassettes, which are inserted in front of the structural gene, have the same effect. By means of inducible promoters it is additionally possible to increase the expression in the course of the production of L-lysine by fermentation. Expression is likewise improved by measures for lengthening the life of the m-RNA, e.g., by inhibiting the rate at which enzyme is degraded. The genes or gene constructs can either be present in plasmids with different copy numbers or be integrated and amplified in the chromosome.

Alternatively, over-expression of the mqo gene can be achieved by changing the composition of the bacterial growth medium and the manner in which culturing is carried out. The person skilled in the art will find a detailed description of procedures that can be followed for carrying out these objectives in a number of publications, including: Martin et al., Bio/Technology 5:137-146 (1987); Guerrero, et al., Gene 138:35-41 (1994); Tsuchiya, et al, Bio/Technology 6:428-430 (1988); Eikmanns, et al., Gene 102:93-98 (1991); EP-B 0 472 869; US 4,601,893; Schwarzer, et al., Bio/Technology 9:84-87 (1991); Reinscheid, et al., Appl. Environment. Microbiol 60:126-132 (1994); LaBarre, et al., J. Bacteriol. 175:1001-1007 (1993); WO 96/15246; Malumbres, et al., Gene 134:15-24 (1993); Jensen et al., Biotech. Bioeng. 58:191-195 (1998); Makrides, Microbiol. Rev. 60:512-538 (1996) and in other standard textbooks of genetics and molecular biology.

An example of a plasmid that can be used in over-expressing malate:quinone oxidoreductase is pRM17 (Molenaar, et al., Eur. J. Biochem. 254:395-403 (1998)). This plasmid is based on the shuttle vector pJC1, which is described in Cremer, et al. (Mol Gen. Genet. 220:478-480). In addition, it may be advantageous for the production of L-amino acids to over-express one or more enzymes of the corresponding biosynthetic pathway as well as malate:quinone oxidoreductase. Thus, for example, in the production of L-lysine, one may also over-express: the dapA gene coding for dihydrodipicolinate synthase (EP-B 0 197 335), or a DNA fragment mediating S-(2-aminoethyl)-cysteine resistance (EP-A 0 088 166). It may also be advantageous for the production of L-amino acids to exclude undesired secondary reactions (see, Nakayama: “Breeding of Amino Acid Producing Microorganisms,” in: Overproduction of Microbial Products, Krumphanzl, Sikyta, Vanek (eds.), Academic Press, London, UK, (1982)).

The microorganisms produced according to the invention may be cultivated continuously or discontinuously in a batch process, in a fed batch, or by a repeated fed batch process for the purpose of producing L-amino acids. A summary of cultivation methods is described in the textbook by Chmiel (Bioprozesstechnik 1. Einfuhrung in die Bioverfahrenstechnik, Gustav Fischer, Verlag, Stuttgart, (1991)) or in the textbook by Storhas (Bioreaktoren und periphere Einrichtungen, Vieweg Verlag, Braunschweig/-Wiesbaden (1994)). The culture medium to be used must meet the requirements of the strain being used for production. Descriptions of culture media for various microorganisms are contained in the handbook Manual of Methods for General Bacteriology of the American Society for Bacteriology, Washington D.C., USA, (1981).

Examples of compounds that can be used as a carbon source include: sugars and carbohydrates such as glucose, saccharose, lactose, fructose, maltose, molasses; starch and cellulose; oils and fats such as soybean oil, sunflower oil, groundnut oil and coconut fat; fatty acids, such as palmitic acid, stearic acid and linoleic acid; alcohols such as glycerol and ethanol; and organic acids such as acetic acid. These substances may be used individually or in the form of a mixture. Examples of compounds that can be used as a nitrogen source include: organic nitrogen-containing compounds such as peptones, yeast extract, meat extract, malt extract, corn steep liquor, soybean flour and urea; or inorganic compounds such as ammonium sulfate, ammonium chloride, ammonium phosphate, ammonium carbonate and ammonium nitrate. The nitrogen sources may be used individually or in the form of a mixture.

Compounds that can be use as a phosphorus source include potassium dihydrogen phosphate and dipotassium hydrogen phosphate or the corresponding sodium-containing salts. The culture medium must also contain salts of metals, such as, for example, magnesium sulfate or iron sulfate, which are necessary for growth. Finally, essential growth substances such as amino acids and vitamins may be used in addition to the above-mentioned substances. Moreover, suitable pre-stages may be added to the culture medium. The mentioned substances may be added to the culture in the form of a single batch or may be fed in a suitable manner during the cultivation.

In order to control the pH of the culture, basic compounds, such as sodium hydroxide, potassium hydroxide, ammonia, or acid compounds, such as phosphoric acid or sulfuric acid, can be used. For controlling the development of foam, antifoams, such as, for example, fatty acid polyglycol esters, may be added.

Plasmid stability can be maintained by adding substances having a selective action, for example antibiotics, to the medium. In order to maintain aerobic conditions, oxygen or oxygen-containing gas mixtures, such as, for example, air, are introduced into the culture.

The temperature of the culture is normally from 20° C. to 45° C. and preferably from 25° C. to 40° C. Culturing is continued until a maximum of the desired L-amino acid has formed. That aim is normally achieved within a period of from 10 hours to 160 hours. Analysis of L-amino acids may be carried out by anion-exchange chromatography with subsequent ninhydrin derivatization, as described by Spackman et al. (Analyt. Chem. 30:1190 (1958)).

The process according to the invention is used for the production of L-amino acids, especially L-aspartic acid, L-asparagine, L-homoserine, L-threonine, L-isoleucine and L-methionine, and especially for the production of L-lysine. The Corynebacterium glutamicum strain DM22/pRM17 was deposited at the Deutsche Sammlung von Mikroorganismen und Zellkulturen (Braunschweig, Germany) under number DSM12711 in accordance with the Budapest Treaty.

The invention may be further understood by reference to the following non-limiting examples.

### EXAMPLES

### Example 1

### Construction of L-lysine Producers Containing Enhanced Malate:Quinone Oxidoreductase

Corynebacterium glutamicum strain DSM5715 (EP-B-0 435 132) was transformed as in Liebl, et al. (FEMS Microbiol. Lett. 65:299-304 (1989)) with the plasmid pRM17 (Molenaar, et al., Eur. J. Biochem. 254: 395-403 (1998)). Selection of the transformants was carried out on LBHIS agar to which 25 mg/l of kanamycin had been added. LBHIS agar consists of LB medium (Sambrook et al., Molecular Cloning: A Laboratory Manual, Cold Spring Harbor Laboratories (1989)) to which there have been added 37 g/l of brain heart bouillon from Merck (Darmstadt, Germany), 0.5 M sorbitol, and 15 g/l of agar-agar. In that manner, the strain DSM5715/pRM17 was formed. The strain DSM5715/pJC1 was produced in the same manner.

### EXAMPLE 2

### Production of L-lysine

The strains DSM5715/pRM17 and DSM5715/pJC1 were first incubated on brain-heart agar, to which kanamycin (25 mg/l) had been added, for 24 hours at 33° C. For cultivation in liquid medium, CgIII medium (Kase, et al., Agri. Biol. Chem. 36:611-1621 (1972)) to which kanamycin (25 mg/l) had additionally been added, was used. To that end, 10 ml of medium, which were contained in 100 ml Erlenmeyer flasks with 4 baffles, were inoculated and the culture was incubated for 16 hours at 240 rpm and 30° C. The culture was subsequently used further as a pre-culture.

The production or test medium used was MM medium, to which kanamycin (25 mg/l) had additionally been added. In the process using strain DSM5715, the corresponding media did not contain kanamycin. The composition and preparation of the MM medium was as follows:

Corn Steep Liquor (CSL): 5 g/l;

3-morpholino-propanesulfonic acid (MOPS): 20g/l;

glucose: 50 g/l (autoclaved separately);

Salts:

(NH4)2SO4): 25 g/l;

KH2PO4: 0.1 g/l;

MgSO4*7H2O: 1.0 g/l;

CaCl2*2H2O: 10 mg/l;

FeSO4*7H2O: 10 mg/l;

MnSO4*H2O: 5.0 mg/l;

biotin: 0.3 mg/l (sterilized by filtration);

thiamine*HCl: 0.2 mg/l (sterilized by filtration);

CaCO3: 25 g/l;

leucine: 0.1 g/l.

CSL, MOPS and the salt solution were adjusted to pH 7 using ammonia water and autoclaved. The sterile substrate and vitamin solutions and the dry autoclaved CaCO₃ were then added.

Cultivation was carried out in 100 ml Erlenmeyer flasks with baffles, which had been charged with 10 ml of the above-described production medium. The cultures were inoculated with the pre-culture so that the optical density at the start was 0.1. Cultivation was carried out at 33° C. and 80% relative humidity.

After incubation for 72 hours, the optical density of the culture suspension and the concentration of L-lysine that had formed were determined. The optical density was determined using an LP2W photometer from Dr. Lange (Berlin, Germany) at a measuring wavelength of 660 nm. L-lysine was determined using an amino acid analyzer from Eppendorf-BioTronik (Hamburg, Germany), by ion-exchange chromatography and post-column reaction with ninhydrin detection. The results are shown in Table 1.

TABLE 1

| | OD | L-lysine |
|---|---|---|
| Strain | (660 nm) | g/l |
| DSM5715 | 10.1 | 16.4 |
| DSM5715/pJC1 | 9.9 | 16.5 |
| DSM5715/pRM17 | 10.2 | 17.8 |

### EXAMPLE 3

### Production of Threonine Producers Containing Enhanced Malate:Quinone Oxidoreductase

Plasmid pRM17 (Molenaar, et al., Eur. J. Biochem. 254:395-403 (1998)) was subjected to electroporation in Corynebacterium glutamicum DSM 5399 by the method of Tauch et al. (FEMS Microbiol. Lett. 123:343-347 (1994)). Strain DSM 5399 is a threonine producer which is described in EP-B-0358940. The selection of transformants was effected by plating out the electroporation batch on LB agar (Sambrook et al., Molecular Cloning: A Laboratory Manual, 2nd ed., Cold Spring Harbor Laboratory Press, Cold Spring Harbor, N.Y., (1989)), to which there had been added 25 mg/l of kanamycin. Strain DSM5399/pRM17 was thus formed.

### EXAMPLE 4

### Production of Threonine

The C. glutamicum strain DSM5399/pRM17 obtained in Example 3 was cultivated in a nutrient medium suitable for the production of threonine, and the threonine content in the culture supernatant was determined. The strain was first incubated on an agar plate with the corresponding antibiotic (brain-heart agar containing kanamycin (25 mg/l)) for 24 hours at 33° C. Starting from the agar plate culture, a pre-culture was inoculated (10 ml of medium in a 100 ml Erlemneyer flask). The medium used for the pre-culture was CgIII complete medium (Kase, et al., Agri. Biol. Chem. 36:1611-1621 (1972)). Kanamycin (25 mg/l) was added thereto. The pre-culture was incubated for 24 hours at 33° C. at 240 rpm in a shaker. A main culture was inoculated from the pre-culture, so that the initial OD (660 nm) of the main culture was 0.1. MM-threonine medium was used for the main culture. Cultivation is carried out in a volume of 10 ml in a 100 ml Erlenmeyer flask with baffles. Kanamycin (25 mg/l) was added. The temperature during cultivation was 33° C. and the relative humidity was 80%.

After 48 hours, the OD was determined at a measuring wavelength of 660 nm using a Biomek 1000 (Beckmann Instruments GmbH, Munich). The concentration of threonine that had formed was determined using an amino acid analyzer from Eppendorf-BioTronik (Hamburg, Germany), by ion-exchange chromatography and post-column derivatization with ninhydrin detection. The results of the test are shown in Table 2.

TABLE 2

| | OD | L-threonine |
|---|---|---|
| Strain | (660 nm) | g/l |
| DSM5399/pRM17 | 13.1 | 0.61 |
| DSM5399 | 13.9 | 0.43 |

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

What is claimed is:

1. In a process for producing L-amino acids by the fermentation of bacteria of the coryneform genus, the improvement comprising enhancing the activity of malate:quinone oxidoreductase in said bacteria.

2. The improvement of claim 1, wherein said activity of malate:quinone oxidoreductase is enhanced by over-expressing the gene encoding this enzyme.

3. The improvement of claim 1, wherein said coryneform bacteria have been treated to enhance the activity of one or more additional enzymes of a synthetic pathway for an L-amino acid.

4. The improvement of claim 1, wherein said coryneform bacteria have been treated to eliminate one or more metabolic pathways that reduce the formation of an L-amino acid.

5. The improvement of any one of claims 1-4, wherein wherein said activity of malate:quinone oxidoreductase is enhanced by transforming said coryneform bacteria with a plasmid vector comprising a nucleotide sequence coding for said malate:quinone oxidoreductase.

6. The improvement according to claim 5, wherein said plasmid vector is pRM17 deposited in Corynebacterium glutamicum, under accession number DSM12711.

7. The improvement of any one of claims 1-4, wherein said process is for the production of an amino acid selected from the group consisting of: L-aspartic acid, L-asparagine, L-homoserine, L-threonine, L-isoleucine and L-methionine.

8. The improvement of claim 7, wherein said process is for the production of L-lysine.

9. The improvement of claim 7, wherein the gene coding for dihydrodipicolinate synthase is over-expressed in said bacteria.

10. The improvement of claim 7, wherein a DNA fragment mediating S-(2-aminoethyl)-cysteine resistance is amplified in said bacteria.

11. A process for producing an L-amino acid by the fermentation of bacteria of the coryneform genus comprising:
a) amplifying the malate:quinone oxidoreductase gene in a bacteria producing said L-amino acid;
b) fermenting the bacteria produced in step a);
c) isolating said L-amino acid made in the fermentation of step b).

12. The process of claim 11, further comprising treating said bacteria to enhance the activity of one or more additional genes of a synthetic pathway for an L-amino acid.

13. The process improvement of claim 11, wherein said bacteria are transformed with plasmid vector pRM17, deposited in Corynebacterium glutamicum, under accession number DSM12711.

14. The process claim 11, wherein said process is for the production of an amino acid selected from the group consisting of: L-aspartic acid, L-asparagine, L-homoserine, L-threonine, L-isoleucine and L-methionine.

15. The process of claim 14, wherein said process is for the production of L-lysine.
